SEQUENCE LISTING (22 sequences)

SEQ ID NO: 1 - protein, 28 aa, Homo sapiens
  LIPID (3)..(3): octanoate
GSSFLSPEHQRVQQRKESKKPPAKLQPR

SEQ ID NO: 2 - protein, 28 aa, Artificial
  ghrelin derivative
XSXXLSPEHQKAQQRKESKKPPAKLQPR

SEQ ID NO: 3 - protein, 18 aa, Artificial
  ghrelin analog
XSXFLSPEHQKAQQRKES

SEQ ID NO: 4 - protein, 28 aa, Artificial
  ghrelin analog
XSXFLSPEHQKAQQRKESKKPPAKLQPR

SEQ ID NO: 5 - protein, 28 aa, Artificial
  ghrelin analog
XSXFLSPEHQKAQQRKESKKPPAKLQPR

SEQ ID NO: 6 - protein, 28 aa, Artificial
  ghrelin analog
XSXXLSPEHQKAQQRKESKKPPAKLQPR

SEQ ID NO: 7 - protein, 28 aa, Artificial
  ghrelin analog
XSXXLSPEHQKAQQRKESKKPPAKLQPR

SEQ ID NO: 8 - protein, 28 aa, Artificial
  ghrelin analog
XSXXLSPEHQKAQQRKESKKPPAKLQPR

SEQ ID NO: 9 - protein, 28 aa, Artificial
  ghrelin analog
XSXXLSPEHQKAQQRKESKKPPAKLQPR

SEQ ID NO: 10 - protein, 28 aa, Artificial
  ghrelin analog
XSXFLSPEHQKAQQRKESKKPPAKLQPR

SEQ ID NO: 11 - protein, 28 aa, Artificial
  ghrelin analog
XSXXLSPEHQKAQQRKESKKPPAKLQPR

SEQ ID NO: 12 - protein, 28 aa, Artificial
  ghrelin analog
XSXXLSPEHQKAQQRKESKKPPAKLQPR

SEQ ID NO: 13 - protein, 18 aa, Artificial
  ghrelin analog
XSXFLSPEHQKAQQRKES

SEQ ID NO: 14 - protein, 18 aa, Artificial
  ghrelin analog
XSXFLSPEHQKAQQRKES

SEQ ID NO: 15 - protein, 18 aa, Artificial
  ghrelin analog
XSXFLSPEHQKAQQRKES

SEQ ID NO: 16 - protein, 28 aa, Artificial
  ghrelin analog
GSXFLSPEHQKAQQRKESKKPPAKLQPR

SEQ ID NO: 17 - protein, 28 aa, Artificial
  ghrelin analog
XSXXLSPEHQKAQQRKESKKPPAKLQPR

SEQ ID NO: 18 - protein, 28 aa, Artificial
  ghrelin analog
XSXXLSPEHQKAQQRKESKKPPAKLQPR

SEQ ID NO: 19 - protein, 28 aa, Artificial
  ghrelin analog
XSXFLSPEHQKAQQRKESKKPPAKLQPR

SEQ ID NO: 20 - protein, 28 aa, Artificial
  ghrelin analog
XSXFLSPEHQKAQQRKESKKPPAKLQPR

SEQ ID NO: 21 - protein, 28 aa, Artificial
  ghrelin analog
XSXXLSPEHQKAQQRKESKKPPAKLQPR

SEQ ID NO: 22 - protein, 18 aa, Artificial
  ghrelin analog
XSXFLSPEHQKAQQRKES

(19) United States
(12) Patent Application Publication
(10) Pub. No.: US 2017/0044230 A1
(43) Pub. Date: Feb. 16, 2017

(54) LONG-ACTING STABLE PEPTIDE GHRELIN ANALOGS FOR THE TREATMENT OF CACHEXIA

(71) Applicants: USTAV ORGANICKE CHEMIE A BIOCHEMIE AV CR, V.V.I., Praha 6 (CZ); FYZIOLOGICKY USTAV AV CR, V.V.I., Praha 4 (CZ)

(72) Inventors: Lenka MALETINSKA, Praha 6 (CZ); Blanka ZELEZNA, Praha 6 (CZ); Jaroslav KUNES, Praha 4 (CZ); Martina HOLUBOVA, Roznov pod Radhostem (CZ); Jana ZEMENOVA, Zliv (CZ)

(73) Assignees: USTAV ORGANICKE CHEMIE A BIOCHEMIE AV CR, V.V.I., Praha 6 (CZ); FYZIOLOGICKY USTAV AV CR, V.V.I., Praha 4 (CZ)

(21) Appl. No.: 15/305,617

(22) PCT Filed: Jun. 22, 2015
(86) PCT No.: PCT/CZ2015/000064

§ 371 Date: Oct. 20, 2016

(30) Foreign Application Priority Data
Jun. 23, 2014 (CZ) PV 2014-429

(51) Int. Cl. C07K 14/575

(52) U.S. Cl. CPC C07K 14/575; A61K 38/00

(57) ABSTRACT

The present invention provides long-acting stable peptide ghrelin analogs of general formulae

(I)
(Sar)S(Dpr-X¹)mLSPEHQKAQQRKESKKPPA(K-Z)LQPR,
and/or
(II)
(Sar)S(Dpr-X²)FLSPEHQKAQQR(K-Z)ES,

in which Dpr is diaminopropionic acid, Sar is sarcosin, X¹ represents a fatty acid residue selected from the group comprising octanoyl, decanoyl, myristoyl, 9-decenoyl and N-10-undecynoyl bound to Dpr through an amide bond, X² represents decanoyl or myristoyl, m represents a non-coded amino acid selected from the group comprising phenylalanine, naphtylalanine, cyclohexylalanine, t-butylalanine and dichlorophenylalanine, Z is palmitoyl which can be optionally bound to the secondary amino group of lysine through an amide bond or Z is not present. The compounds of the invention are suitable for use in a method of treatment of cachexia and/or anorexia.

### FIELD OF THE ART

Stable agonists of gastrointestinal hormone ghrelin represent orexigenic compounds which increase food intake after peripheral administration. Therefore ghrelin agonist could be potentially useful for treatment of cachexia or anorexia. We describe synthesis of the peptide analogs and their pharmacological properties both in vitro and in vivo.

### STATE OF THE ART

Cachexia is characterized by physical wasting and by loss of muscle mass with or without loss of fat mass. This state represents a result of catabolic/anabolic imbalance and it often accompanies advanced cancer or chronic progressive diseases. In patients suffering from these diseases, cachexia and anorexia often co-exist and induce malnutrition in 20% of patients with congestive heart failure, 20% of patients with chronic obstructive pulmonary disease, 40% of patients with renal failure, 30% onkologic patients and 85% of patients with gastrointestinal tumors. The major mechanisms contributing to the development of cachexia are chronic inflammation, elevated levels of circulating cytokines and increase in activity of the sympathetic nervous system. The last one, together with elevated plasmatic levels of catecholamines, contributes to systemic hypermetabolism followed by malnutrition and increase in energy expenditure. Cachexia is usually connected with worsened prognosis of the underlying disease and it contributes to the increase in the mortality rate. Overcoming of cachexia could reduce the treatment costs and improve quality of life in oncologic and chronically ill patients. However, potential pharmacological interventions are limited so far; they include appetite stimulants, anabolic steroids or cytokine modulators. Lately, ghrelin and other agonists of ghrelin receptor (GHS-R1a) have gained attention as a potential treatment for cachexia.

Ghrelin, the only known orexigenic gut hormone, is secreted predominantly from the stomach. Ghrelin secretion increases with fasting and before meal initiation and falls to trough levels within 1 h after meal. Besides the stomach, ghrelin is also expressed in other tissues, among others in the hypothalamic neurons (Depoortere, 2009).

Ghrelin is a 28-amino-acid peptide (H-Gly-Ser-Ser(octanoyl)-Phe-Leu-Ser-Pro-Glu-His-Gln-Arg-Val-Gln-Gln-Arg-Lys-Glu-Ser-Lys-Lys-Pro-Pro-Ala-Lys-Leu-Gln-Pro-Arg-OH) (SEQ ID NO. 1). Hydroxyl group of the Ser³ at the N-terminus is esterified by n-octanoic acid. This unique modification is necessary for biological activity of ghrelin (Kojima et al., 1999). Structure-function studies in vitro in cells overexpressing ghrelin receptor revealed that N-terminal tetrapeptide octanoylated at Ser³ represents the minimal active core preserving most of in vitro biological activity of full length ghrelin molecule (Matsumoto et al., 2001). N-terminal positive charge and Phe⁴ were shown to be essential for biological activity of ghrelin (Van Craenenbroeck et al., 2004). Ester bond in Ser³-O-octanoyl is an easy subject to hydrolysis, therefore only 10-20% of circulating ghrelin is octanoylated and its half-life in blood is only a few minutes. The role of des-octanoyl ghrelin is still unclear.

Ghrelin receptor (GHS-R1a) was initially described as an orphan receptor of synthetic growth hormone secretagogues (GHS) (Howard et al., 1996). Later, ghrelin was identified as a natural ligand of GHS-R1a and as an endogenous mediator of growth hormone (GH) release (Kojima et al., 1999). GHS-R1 a is predominantly expressed in the arcuate nucleus (ARC) of the hypothalamus, where it increases food intake after interaction with ghrelin. Other expression sites of GHS-R1a include the pituitary somatotroph cells, where the interaction of ghrelin with GHS-R1a stimulates GH release, and cells of the immune system, where GHS-R1 a mediates regulation of the immune response (Hosomi et al., 2008).

Ghrelin was shown to maintain positive energy balance of the organism. It increases food intake and subsequently body weight, it facilitates adipose tissue accumulation and also attenuates spontaneous physical activity (Nakazato et al., 2001). Besides the central orexigenic effect of ghrelin, its anti-inflammatory and cardiovascular effects can be also beneficial for improvement of the cachectic state. Ghrelin's potent effect on fat storage can provide important energy reserves during the continuing processes of cachexia (DeBoer, 2011).

Clinical trials showed that intravenous administration of synthetic human ghrelin to cachectic patients is safe and that ghrelin effectively increases appetite in cachectic patients (Strasser et al., 2008).

Up to now, a number of both peptide and non-peptide GHS-R1a agonists were described. Some of them were, similarly to ghrelin, tested as promising anti-cachectic therapeutics. Compounds BIM-28125 and BIM-28131 were tested in rat models of cancer-induced and renal cachexia (Deboer et al., 2008), peptides GHRP-1, GHRP-2, GHRP-6 and hexarelin were tested in rat model of cardiac cachexia (Xu et al., 2005). Anamorelin was employed in pilot study in cachectic oncologic patients (Garcia et al., 2013), MK-677 was tested in healthy volunteers aged 61-80 years (Nass et al., 2008). JMV1843 (macimorelin) is in ongoing phase II trial for its use for treatment of cancer-induced cachexia (Aeterna Zentaris; clinicaltrials.gov; NCT01614990).

### DISCLOSURE OF THE INVENTION

We prepared long-acting, stable peptide analogs of ghrelin with modified amino acid sequence, which are lipidized by a fatty acid in position 3 or in some cases by a second fatty acid in position 16 or 24. These analogs bind to GHS-R1a receptor with high affinity in in vitro conditions and show a statistically significant, dose-dependent increase in food intake after the peripheral (subcutaneous) administration to fed mice.

Thus, the object of the invention is represented by 28-amino-acid peptides derived from the ghrelin sequence, wherein the N-terminal glycine is replaced by sarcosine, serine bound to the n-octanoyl through the ester bond in position 3 is replaced by diaminopropionic acid bound to a fatty acid residue selected from the group comprising octanoyl, decanoyl, myristoyl, 9-decenoyl, N-10-undecynoyl, through the amide bond. Phenylalanine in position 4 can be replaced by a non-coded amino acid selected from the group comprising β-cyclohexylalanine, L-1-naphtylalanine, t-butylalanine and dichlorophenylalanine. The secondary amino group of Lys²⁴ may optionally be acylated by palmitic acid. The object of the invention is further represented by 18-amino-acid peptides (starting from N-terminus of ghrelin sequence), where the N-terminal glycine is replaced by sarcosine, serine bound to the n-octanoyl through the ester bond in position 3 is replaced by diaminopropionic acid bound to decanoyl or myristoyl through the amide bond, and secondary amino group of Lys¹⁶ may optionally be acylated by palmitic acid.

The peptides of the invention are compounds of general formulae,

(I)
(SEQ ID NO. 2)
(Sar)S(Dpr-X¹)mLSPEHQKAQQRKESKKPPA(K-Z)LQPR,
and/or
(II)
(SEQ ID NO. 3)
(Sar)S(Dpr-X²)FLSPEHQKAQQR(K-Z)ES,

where Dpr stands for diaminopropionic acid, Sar stands for sarcosin, X¹ represents a fatty acid residue selected from the group comprising octanoyl (oct), decanoyl (dec), myristoyl (myr), 9-decenoyl (decen) and N-10-undecynoyl (undec) bound to Dpr through an amide bond, X² represents decanoyl or myristoyl, m represents a non-coded amino acid selected from the group comprising phenylalanine, naphtylalanine (Nal), cyclohexylalanine (Cha), t-butylalanine and dichlorophenylalanine (PheCl₂), Z is palmitoyl which can be optionally bound to the secondary amino group of lysine through an amide bond or Z is not present.

More particularly, the invention includes long-acting stable peptide ghrelin analogs of the general formulae I and/or II, selected from the group comprising:

(SEQ ID NO. 4)
(Sar)S(Dpr-N-dec)FLSPEHQKAQQRKESKKPPAKLQPR
(SEQ ID NO. 5)
(Sar)S(Dpr-N-myr)FLSPEHQKAQQRKESKKPPAKLQPR
(SEQ ID NO. 6)
(Sar)S(Dpr-N-dec)(1-Nal)LSPEHQKAQQRKESKKPPAKLQPR
(SEQ ID NO. 7)
(Sar)S(Dpr-N-myr)(1-Nal)LSPEHQKAQQRKESKKPPAKLQPR
(SEQ ID NO. 8)
(Sar)S(Dpr-N-dec)(Cha)LSPEHQKAQQRKESKKPPAKLQPR
(SEQ ID NO. 9)
(Sar)S(Dpr-N-myr)(Cha)LSPEHQKAQQRKESKKPPAKLQPR
(SEQ ID NO. 10)
(Sar)S(Dpr-N-oct)FLSPEHQKAQQRKESKKPPAK(N-palm)
LQPR
(SEQ ID NO. 11)
(Sar)S(Dpr-N-oct)(Cha)LSPEHQKAQQRKESKKPPAK(N-palm)
LQPR
(SEQ ID NO. 12)
(Sar)S(Dpr-N-myr)(PheCl₂)LSPEHQKAQQRKESKKPPAKLQPR
(SEQ ID NO. 13)
(Sar)S(Dpr-N-dec)FLSPEHQKAQQRK(N-palm)ES
(SEQ ID NO. 14)
(Sar)S(Dpr-N-myr)FLSPEHQKAQQRK(N-palm)ES
(SEQ ID NO. 15)
(Sar)S(Dpr-N-dec)FLSPEHQKAQQRKES

Another aspect of the present invention are the long-acting stable peptide ghrelin analogs for use as a medicament, in particular for treating cachexia and/or anorexia.

A further aspect of the invention are the long-acting stable peptide ghrelin analogs for use as an orexigenic compounds for increasing food intake after peripheral administration.

Another aspect of the invention is a pharmaceutical composition which contains at least one long-acting stable peptide ghrelin analog as an active compound. It may also contain further active compounds and/or pharmaceutically acceptable auxiliary substances.

Yet another aspect of the invention is represented by use of the long-acting stable peptide ghrelin analogs in manufacture of a medicament for treatment of cachexia or anorexia.

The examples below show the following properties and effects of the ghrelin analogs of the present invention, inter alia:
Ghrelin analogs were found to possess affinities similar to those of ghrelin/Dpr³ ghrelin for cell membranes with transfected GHS-R1a.
Ghrelin analogs induced intracellular calcium mobilization and activated inositolphosphate cascade comparably to ghrelin/Dpr³ ghrelin.
Ghrelin analogs dose-dependently and significantly more potently increased food intake after peripheral (subcutaneous) administration to mice.
The effect of ghrelin analogs on food intake was long-term, lasting up to 10 hours after administration depending on dose. This fact was probably caused by enhanced resistance of the analogs against degradation by proteases, which was enabled by the incorporation of the specific alternative fatty acid and non-coded amino acids.
Stability of the new ghrelin analogs in vitro in rat plasma and in vivo in mice after SC administration was significantly higher when compared to ghrelin/Dpr³ ghrelin.
Repeated subcutaneous administration of the specific analog (Sar)S(Dpr-N-myr)FLSPEHQKAQQRKESKKPPAKLQPR (analog no. 2) improved the cachectic state in the rat model of subtotal nephrectomy: it increased body weight and reduced blood levels of pro-inflammatory cytokines.
28-day continuous intraperitoneal infusion of the specific analog (Sar)S(Dpr-N-myr)FLSPEHQKAQQRKESKKPPAKLQPR (analog no. 2) improved the cachectic state in the rat model of aortic stenosis-induced cachexia: it significantly increased body weight.
A single subcutaneous administration of the selected potent analog (Sar)S(Dpr-N-myr)(PheCl₂)LSPEHQKAQQRKESKKPPAKLQPR (analog no. 14) increased food intake in the mouse model of lipopolysaccharide-induced cachexia/sepsis, reduced plasmatic levels of pro-inflammatory cytokines, increased expression of orexigenic neuropeptides in the hypothalamus and tended to decrease expression of markers of muscle degradation.

According to in vivo testing of the effect of the ghrelin analogs on food intake, the following analogs were the most potent ones:

No. 1
(SEQ ID NO. 4)
(Sar)S(Dpr-N-dec)FLSPEHQKAQQRKESKKPPAKLQPR
No. 2
(SEQ ID NO. 5)
(Sar)S(Dpr-N-myr)FLSPEHQKAQQRKESKKPPAKLQPR
No. 3
(SEQ ID NO. 6)
(Sar)S(Dpr-N-dec)(1-Na1)LSPEHQKAQQRKESKKPPAKLQPR
No. 4
(SEQ ID NO. 7)
(Sar)S(Dpr-N-myr)(1-Nal)LSPEHQKAQQRICESKKPPAKLQPR
No. 5
(SEQ ID NO. 8)
(Sar)S(Dpr-N-dec)(Cha)LSPEHQKAQQRKESKKPPAKLQPR
No. 6
(SEQ ID NO. 9)
(Sar)S(Dpr-N-myr)(Cha)LSPEHQKAQQRKESKKPPAKLQPR
No. 7
(SEQ ID NO. 10)
(Sar)S(Dpr-N-oct)FLSPEHQKAQQRKESKKPPAK(N-palm)LQPR
No. 8
(SEQ ID NO. 11)
(Sar)S(Dpr-N-oct)(Cha)LSPEHQKAQQRKESKKPPAK
(N-palm)LQPR
No. 14
(SEQ ID NO. 12)
(Sar)S(Dpr-N-myr)(PheCl₂)LSPEHQKAQQRKESKKPPAKLQPR
No. 15
(SEQ ID NO. 13)
(Sar)S(Dpr-N-dec)FLSPEHQKAQQRK(N-palm)ES
No. 16
(SEQ ID NO. 14)
(Sar)S(Dpr-N-myr)FLSPEHQKAQQRK(N-palm)ES
No. 17
(SEQ ID NO. 15)
(Sar)S(Dpr-N-dec)FLSPEHQKAQQRKES.

### BRIEF DESCRIPTION OF THE DRAWINGS

FIG. 1 shows orexigenic effect (cummulative food intake in dependence on time) of selected ghrelin analogs no. 1, 2, 3, 4, 5 and 6 after their Sc administration to fed C57BL/6 male mice at a dose of 5 mg/kg of body weight. Significance: ** P<0.01, *** P<0.001 vs. saline (one-way ANOVA followed by Dunnett's post-hoc test), n=6-8.

FIG. 2 shows orexigenic effect (cummulative food intake in dependence on time) of selected ghrelin analogs no. 14, 15, 16, 17 and 18 after their SC administration to fed C57BL/6 male mice at a dose of 5 mg/kg of body weight. Significance: *P<0.05, ** P<0.01, *** P<0.001 vs. saline (one-way ANOVA followed by Dunnett's post-hoc test), n=5-6.

FIG. 3 shows dose-dependent orexigenic effect (cummulative food intake in dependence on time) of myristoylated analog [Sar¹, Dpr(myr)³, 1-Nal⁴]ghrelin (analog no. 4) after its SC administration to fed C57BL/6 male mice at doses of 0.2, 1, 5 and 10 mg/kg of body weight. Significance: *** P<0.001 vs. saline (one-way ANOVA followed by Dunnett's post-hoc test), n=5.

FIG. 4 shows pharmacokinetics of ghrelin, Dpr³ghrelin and selected ghrelin analogs no. 4, 6, 14 and 18 (concentration of analogs in blood in dependence on time) after their SC administration to C57BL/6 male mice at a dose of 5 mg/kg of body weight. n=4.

FIG. 5 shows growth hormone release 10 min after SC administration of ghrelin, Dpr³ghrelin and selected ghrelin analogs no. 4 and 14 at a dose of 5 mg/kg to C57BL/6 male mice aged 4-5 weeks. Significance: *P<0.05, ** P<0.01 vs. saline (one-way ANOVA followed by Dunnett's post-hoc test), n=4-5.

FIG. 6 shows body weight change after repeated (15-day) SC administration of analog no. 2 (5 mg/kg of body weight) to the rat model of subtotal nephrectomy (SNx). Significance: * P<0.05, *** P<0.001 vs. control group SNx/saline (one-way ANOVA followed by Dunnett's post-hoc test), n=5-7.

FIG. 7 shows body weight change after 28-day continuous IP infusion of analog no. 2 to the rat model of aortic stenosis-induced cachexia. Significance: *P<0.05, ** P<0.01 vs. saline (t-test), n=5.

FIG. 8 shows the increase in food intake after the SC administration of analog no. 14 (5 mg/kg of body weight) to the mouse model of lipopolysaccharide-induced cachexia (LPS model). Mice were injected with LPS or saline at 17:00 and with ghrelin analog or saline at 7:00 the next day, their food intake was monitored for 10 hours. Significance: *** P<0.001 vs. control group LPS+saline (one-way ANOVA followed by Dunnett's post-hoc test), n=4-5.

FIG. 9 shows levels of C-reactive protein (a) and pro-inflammatory cytokine IL-2 (b) after the SC administration of analog no. 14 (5 mg/kg of body weight) to the mouse model of lipopolysaccharide-induced cachexia (LPS model). Significance: *P<0.05, ** P<0.01 (one-way ANOVA followed by Bonferroni's post-hoc test), n=4-5.

FIG. 10 shows expression of mRNA for orexigenic neuropeptides NPY (a) and AgRP (b) in hypothalamus after the SC administration of analog no. 14 (5 mg/kg of body weight) to the mouse model of lipopolysaccharide-induced cachexia (LPS model). Significance: *** P<0.001 (one-way ANOVA followed by Bonferroni's post-hoc test), n=4-5.

FIG. 11 shows expression of mRNA for markers of muscle degradation MuRF-1 (a) and MAFbx (b) after the SC administration of analog no. 14 (5 mg/kg of body weight) to the mouse model of lipopolysaccharide-induced cachexia (LPS model). Significance: *** P<0.001 (one-way ANOVA followed by Bonferroni's post-hoc test), n=4-5.

### EXAMPLES

Abbreviations

AgRP agouti-related peptide

ANOVA analysis of variance

AS aortic stenosis

BSA bovine serum albumine

BPTI bovine pancreatic trypsin inhibitor

CRP C-reactive protein

GAPDH glyceraldehyde 3-phosphate dehydrogenase

GH growth hormone

GHS-R1a growth hormone secretagogue receptor 1a

GUSB beta glucuronidase

HBSS Hank's balanced salt solution

Hepes 4-(2-hydroxyethyl)-1-piperazineethanesulfonic acid

HTRF homogeneous time resolved fluorescence

IL-2 interleukin 2

IP intraperitoneal

LPS lipopolysaccharide

MAFbx muscle atrophy F-box

MuRF-I muscle ring finger I

NPY neuropeptide Y

PEI polyethylenimine

SC subcutaneous

SNx subtotal (5/6) nephrectomy

Tris tris(hydroxymethyl)aminomethane

Material and Methods Used for Tests with Ghrelin Analogs

Peptides were assembled in a solid-phase ABI 433A synthesizer (Applied Biosystems, Foster City, Calif., USA) by stepwise coupling of the corresponding Fmoc amino acids to the growing chain according to the procedure described by Maixnerová and co-workers (Maixnerová et al., 2007). Lipidization with the corresponding fatty acid was performed before cleavage of the peptide from the resin according to the procedure described by Maletinská and co-workers (Maletinská et al., 2012).

Ghrelin was iodinated at His⁹ with Na¹²⁵I using Iodo-Gen (Pierce, Rockford, Ill., USA) according to the protocol recommended by the manufacturer. Monoiodinated peptide was stored in aliquots at −20° C. and used for binding studies within 1 month.

### Example 1

### Competitive Binding Studies

Competitive binding studies were performed according to the principles of Motulsky and Neubig (Motulsky & Neubig, 2002). Isolated plasma membranes from the HEK293T cells with transfected human GHS-R1a receptor (Multispan, Hayward, Calif., USA) were used. Incubations were performed in a total volume of 0.25 ml of binding buffer (50 mM Tris pH 7.4, 5 mM MgCl₂, 2.5 mM EDTA, 1 mg/ml BSA, 0.1 mg/ml BPTI) for 45 mM at 25° C., with 0.05 nM of ¹²⁵I-ghrelin and 1 pM to 10 μM of nonradioactive ghrelin/ghrelin analog. The binding reaction was stopped by the addition of ice-cold washing buffer (20 mM Tris pH 7.4, 10 mM MgCl₂, 2.5 mM EDTA, 0.015% Triton X-100) followed by rapid filtration over GF/C filters (Whatman, Clifton, N.J., USA) presoaked with 0.5% PEI in binding buffer using a Brandel cell harvester (Brandel Inc., Gaithersburg, Md., USA). Bound radioactivity was determined by gamma counting (Wizard 1470 Automatic Gamma Counter; PerkinElmer Life and Analytical Sciences, Waltham, Mass.).

Experiments were carried out in duplicates at least three times.

Competitive binding curves were plotted using GraphPad Prism Software (San Diego, Calif., USA) while comparing the best fit for single binding sites models. IC₅₀ values were obtained from nonlinear regression analysis, IC, values (inhibition constants) were calculated from IC₅₀ values using the Cheng-Prusoff equation (Cheng and Prusoff, 1973) and the K_{d} value 0.1938 nM which was obtained from saturation binding experiments.

All tested ghrelin analogs, whose structures are listed in Tab. 1, showed high affinities for cell membranes with over-expressed GHS-R1a and had Kᵢ values in nanomolar or even lower range. Analogs no. 1, 3, 5, 11 showed higher affinities for GHS-R1a than ghrelin. Kᵢ values are summarized in Tab. 2.

TABLE 1
Structures of ghrelin analogs

| Ghrelin | GS(S-oct)FLSPEHQKAQQRKESKKPPAKLQPR (SEQ ID NO. 1) |
|---|---|
| Dpr³ghrelin | GS(Dpr-oct)FLSPEHQKAQQRKESKKPPAKLQPR (SEQ ID NO. 16) |
| 1 | (Sar)S(Dpr-N-dec)FLSPEHQKAQQRKESKKPPAKLQPR (SEQ ID NO. 4) |
| 2 | (Sar)S(Dpr-N-myr)FLSPEHQKAQQRKESKKPPAKLQPR (SEQ ID NO. 5) |
| 3 | (Sar)S(Dpr-N-dec)(1-Nal)LSPEHQKAQQRKESKKPPAKLQPR (SEQ ID NO. 6) |
| 4 | (Sar)S(Dpr-N-myr)(1-Nal)LSPEHQKAQQRKESKKPPAKLQPR (SEQ ID NO. 7) |
| 5 | (Sar)S(Dpr-N-dec)(Cha)LSPEHQKAQQRKESKKPPAKLQPR (SEQ ID NO. 8) |
| 6 | (Sar)S(Dpr-N-myr)(Cha)LSPEHQKAQQRKESKKPPAKLQPR (SEQ ID NO. 9) |
| 7 | (Sar)S(Dpr-N-oct)FLSPEHQKAQQRKESKKPPAK(N-palm)LQPR (SEQ ID NO. |
| | 10) |
| 8 | (Sar)S(Dpr-N-oct)(Cha)LSPEHQKAQQRKESKKPPAK(N-palm)LQPR (SEQ ID |
| | NO. 11) |
| 9 | (Sar)S(Dpr-N-oct)(Nal)LSPEHQKAQQRKESKKPPAK(N-palm)LQPR (SEQ ID |
| | NO. 17) |
| 10 | (Sar)S(Dpr-N-oct)(tBu)LSPEHQKAQQRKESKKPPAKLQPR (SEQ ID NO. 18) |
| 11 | (Sar)S(Dpr-N-10-undecyn)FLSPEHQKAQQRKESKKPPAKLQPR (SEQ ID NO. |
| | 19) |
| 12 | (Sar)S(Dpr-N-9-decen)FLSPEHQKAQQRKESKKPPAKLQPR (SEQ ID NO. 20) |
| 13 | (Sar)S(Dpr-N-oct)(PheCl₂)LSPEHQKAQQRKESKKPPAKLQPR (SEQ ID NO. |
| | 21) |
| 14 | (Sar)S(Dpr-N-myr)(PheCl₂)LSPEHQKAQQRKESKKPPAKLQPR (SEQ ID NO. |
| | 12) |
| 15 | (Sar)S(Dpr-N-dec)FLSPEHQKAQQRK(N-palm)ES (SEQ ID NO. 13) |
| 16 | (Sar)S(Dpr-N-myr)FLSPEHQKAQQRK(N-palm)ES (SEQ ID NO. 14) |
| 17 | (Sar)S(Dpr-N-dec)FLSPEHQKAQQRKES (SEQ ID NO. 15) |
| 18 | (Sar)S(Dpr-N-myr)FLSPEHQKAQQRKES (SEQ ID NO. 22) |

Oct-octanoyl, Dpr-diaminopropionic acid, Sar-sarcosine, dec-decanoyl, myr-myristoyl, 1-Nal-naphtylalanine, Cha-cyclohexylalanine, tBu-tertbutylalanine, 10-undecyn-undecynoyl, 9-decen-decenoyl, PheC12-dichlorophenylalanine

TABLE 2
Affinities of ghrelin analogs for GHS-R1a receptor
(competitive displacement of ¹²⁵I-ghrelin
binding by peptide ghrelin analogs)

| Analog | Kᵢ [nM] | % of ghrelin binding |
|---|---|---|
| Ghrelin | 1.15 ± 0.20 | 100 |
| Dpr³ ghrelin | 0.68 ± 0.02 | 169 |
| 1 | 0.63 ± 0.03 | 183 |
| 2 | 2.10 ± 0.16 | 54 |
| 3 | 0.65 ± 0.07 | 176 |
| 4 | 2.01 ± 0.11 | 57 |
| 5 | 1.04 ± 0.06 | 110 |
| 6 | 6.41 ± 0.51 | 18 |
| 7 | 6.09 ± 0.74 | 19 |
| 8 | 20.8 | 6 |
| 9 | 23.3 ± 5.34 | 5 |
| 10 | 4.71 ± 0.45 | 24 |
| 11 | 0.83 ± 0.02 | 139 |
| 12 | 1.51 ± 0.43 | 76 |
| 13 | 2.45 ± 0.37 | 47 |
| 14 | 16.8 ± 1.81 | 7 |
| 15 | 5.99 ± 0.79 | 19 |
| 16 | 51 ± 8.65 | 2 |
| 17 | 3.71 ± 0.78 | 30 |
| 18 | 3.38 ± 0.90 | 34 |

### Example 2

### Cell Signalling—Functional Studies

Inositol phosphate (IP1) accumulation was determined using the IP-One HTRF assay kit (Cisbio Bioassays) according to the protocol recommended by the manufacturer. HEK-293T cells transiently transfected with the human GHS-R1a receptor were cultured at 96-well assay plates (seeded at 50 000 cells/well). 48 h after transfection, the cells were stimulated with tested ligands at concentrations from 10 pM to 10 μM in cell stimulation buffer (10 mMHepes, 1 mM CaCl₂, 0.5 mM MgCl₂, 4.2 mM KCl, 146 mM NaCl, 5.5 mM glucose, 50 mM LiCl, pH 7.4) for 45 min at 37° C. in duplicates.

Intracellular calcium mobilization assay was performed according to the described procedure (Demange et al., 2007). HEK-293T cells were transiently transfected with the human GHS-R1a receptor and were plated into 96-well black-bottom plates (80 000 cells/well). 24 hours later (after the cells reached 80-95% confluence) the cells were washed with 150 _{R}I reaction buffer (HBSS, 0.5% BSA, 20 mM HEPES, 1 mM MgSO₄, 1.3 mM CaCl₂, pH 7.4) and were than loaded with 1 μM fluorescent calcium indicator Fluo-4AM prepared in reaction buffer containing 0.06% pluronic acid. The cells were incubated for 1 h in the dark at 37° C. Following the incubation, excess Fluo-4AM was removed by washing twice with 100 μl of reaction buffer, and 50 μl of the reaction buffer was added to each well. The experiment was performed using the benchtop scanning fluorometer FlexStation II, the tested analogs were automatically added to the cells in concentrations from 10 pM to 10 μM in triplicates.

EC₅₀ values were calculated using the GraphPad Prism Software (San Diego, Calif., USA) and are summarized in Tab. 3.

TABLE 3
Inositol phosphate (IP1) acummulation
and intracellular Ca²⁺ mobilization

| Analog | IP1 - EC₅₀ [nM] | Ca²⁺ - EC₅₀ [nM] |
|---|---|---|
| Ghrelin | Not tested (NT) | 3.32 |
| Dpr³ ghrelin | 16.3 | NT |
| 1 | 12.6 | 9.66 |
| 2 | 7.10 | 36.6 |
| 3 | 2.08 | 18.6 |
| 4 | 9.75 | 10.9 |
| 5 | 3.28 | 10.6 |
| 6 | 3.08 | 11.4 |

### Example 3

### Effect of SC Administered Ghrelin Analogs on Food Intake

All of the experiments followed the ethical guidelines for animal experiments and Czech Republic law 246/1992 and were approved by the committee for experiments with laboratory animals of the Academy of Sciences of the Czech Republic. Male C57BL/6 mice (Charles River, Germany) were housed at a temperature 22±2° C. under a daily cycle of 12/12 h light/dark (light from 6:00) with free access to water and a standard chow diet St-1 which contained 66%, 25% and 9% of calories from protein, fat and carbohydrate, respectively, and its energy content was 3.4 kcal/g (Mlýn Kocanda, Praha, Czech Republic). Mice were placed into separate cages for one week before experiment, they had free access to water and food pellets. Closely before the experiment, the food pellets were removed from the cages. At 8:00 a.m., mice were injected subcutaneously with 0.2 ml of saline, ghrelin or ghrelin analogs (dissolved in saline) at doses of 0.1-10 mg/kg of body weight. 15 min after the injection, mice were given preweighed food pellets. Food intake was monitored for 8-10 hours at 30-min intervals. Mice had free access to water during the experiment. Experiments were performed at least twice for each tested compound, each experimental group had at least 5 mice.

Data from food intake experiments were analyzed by one-way ANOVA followed by Dunnett's post-hoc test using GraphPad Prism Software (San Diego, Calif., USA), P<0.05 was considered statistically significant. ED₅₀ values were calculated using GraphPad Prism Software as the dose of the tested compound required to elicit half-maximal effect at 250 min after administration of the corresponding compound.

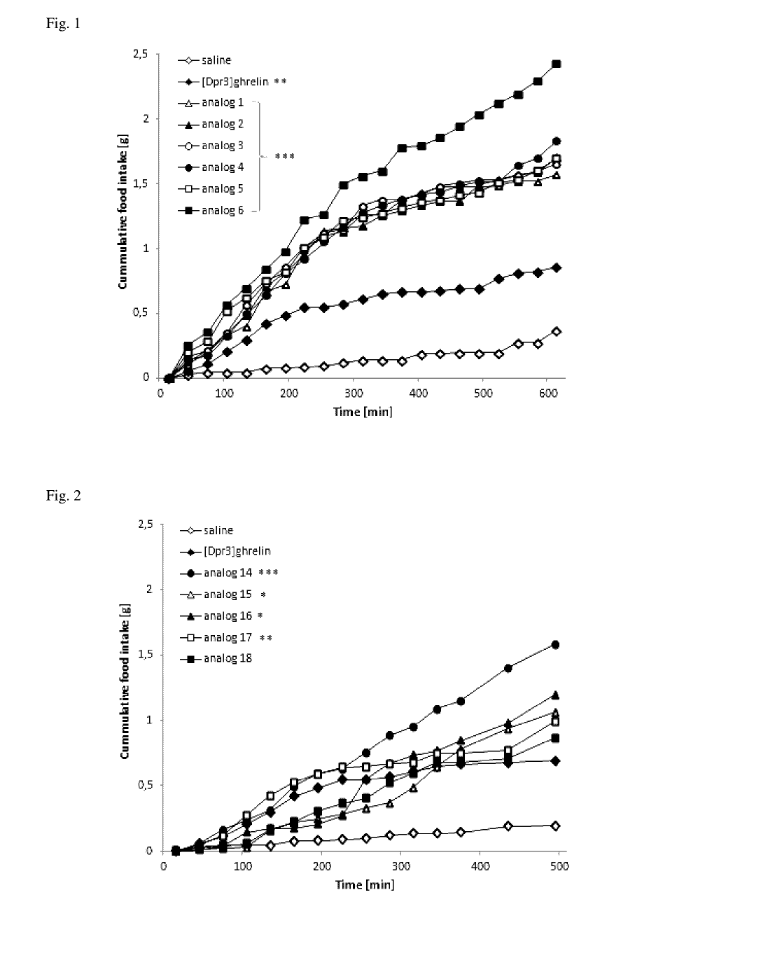

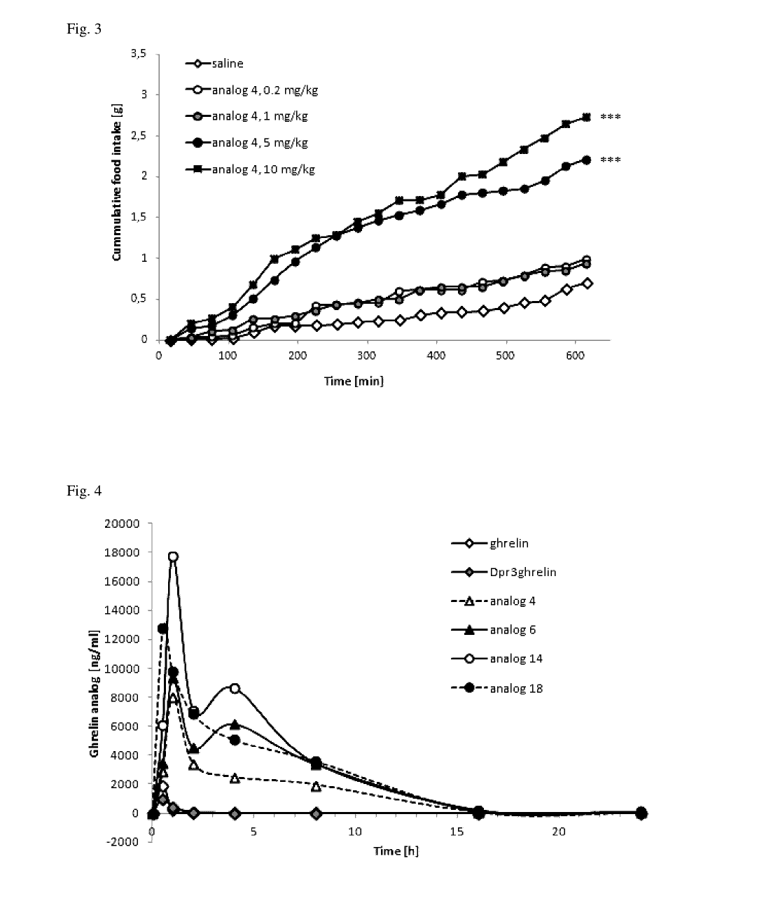

Tab. 4 summarizes ED₅₀ values for particular analogs and maximal effect on food intake at a dose of 5 mg/kg of body weight related to the maximal effect of Dpr³ghrelin. FIGS. 1 a 2 show increase in food intake after administration of selected potent ghrelin analogs to mice. The effect on food intake was dose-dependent (FIG. 3).

TABLE 4
Biological activity of ghrelin analogs in vivo - effect on food intake

| | ED₅₀ [mg/kg] | maximal effect at 5 mg/kg |
|---|---|---|
| | (250 min | [% of Dpr³ghr] |
| Analog | after injection) | [symbol] |
| Ghrelin | 2.39 | 106.5 |
| Dpr³ ghrelin | 0.82 | 100 |
| 1 | 3.90 | 218.8 |
| 2 | 3.80 | 202.7 |
| 3 | 0.64 | 220.3 |
| 4 | 6.02 | 212.8 |
| 5 | 1.02 | 204.5 |
| 6 | 3.05 | 276.2 |
| 7 | 9.98 | 193.9 |
| 8 | NT | 200.2 |
| 9 | NT | 171.1 |
| 10 | 0.85 | 122.7 |
| 11 | 6.62 | 124.8 |
| 12 | NT | 141.5 |
| 13 | NT | 92.9 |
| 14 | 0.65 | 235.5 |
| 15 | NT | 158.8 |
| 16 | NT | 177.6 |
| 17 | NT | 147.9 |
| 18 | NT | 128.5 |

[symbol]

indicates data missing or illegible when filed

Food intake was monitored for 480-600 mm after administration of the compounds. ED₅₀ values were determined 250 min after administration. Maximal effect on food intake was evaluated 480 mm after administration of the compounds at a dose of 5 mg/kg of body weight. NT—not tested.

### Example 4

### Stability of Selected Ghrelin Analogs in Rat Plasma In Vitro and Pharmacokinetics of Selected Ghrelin Analogs In Vivo After the SC Administration to Mice

Stability of ghrelin analogs (Dpr³ghrelin and analogs no. 2, 14 and 18) was tested in vitro by incubation of the compounds at a concentration of 1 μM in rat plasma at 37° C. for various time periods (0-24 h). Subsequently, the samples were taken from the plasma pool and the incubation was stopped by quick freezing to −20° C. Concentration of ghrelin analogs in samples was determined by Rat/Mouse Ghrelin ELISA kit (Merck-Millipore, St. Charles, Mo., USA), corresponding ghrelin analog was used as a calibration standard.

All the tested ghrelin analogs were found to be highly stable, with half-life in plasma longer than 24 h. For comparison, half-life of acylated ghrelin in plasma is approximately 10 min. Pharmacokinetics of ghrelin analogs was tested in C57BL/6 male mice (Charles River, Germany). Mice were housed at a temperature 22±2° C. under a daily cycle of 12/12 h light/dark (light from 6:00) with free access to water and a standard chow diet St-1 (Mlýn Kocanda, Praha, Czech Republic).

Mice were SC injected with ghrelin, Dpr³ghrelin or one of the selected ghrelin analogs no. 4, 6, 14 and 18 at a dose of 5 mg/kg of body weight (0.2 ml/mouse, n=4). Blood was collected from tails before injection and 0.5, 1, 2, 4, 8, 16 and 24 h after injection. Plasma was separated and stored at −20° C. Concentration of ghrelin analogs in samples was determined by Rat/Mouse Ghrelin ELISA kit (Merck-Millipore, St. Charles, Mo., USA), corresponding ghrelin analog was used as a calibration standard.

Ghrelin analogs no. 4, 6, 14 and 18 were significantly more stable than ghrelin or Dpr³ghrelin. Half-life of analogs 6 and 14 was 7 and 5 h, respectively (FIG. 4).

### Example 5

### Growth Hormone (GH) Release After SC Administration of Selected Ghrelin Analogs to Young Mice

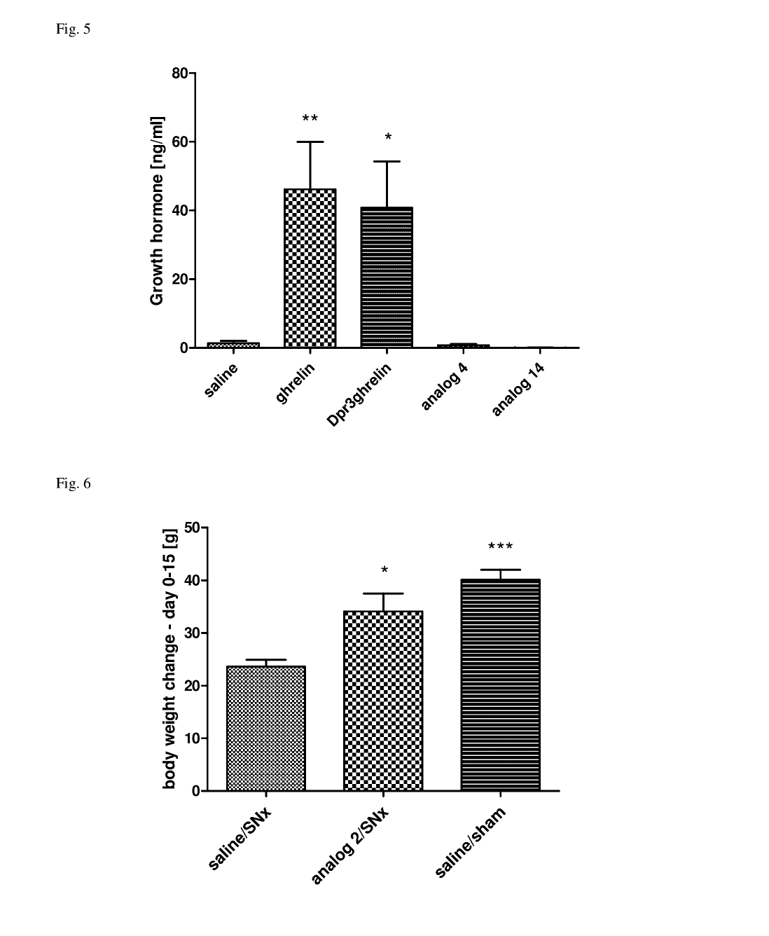

The effect of selected ghrelin analogs on GH release was determined in 4-5-week old male C57BL/6 mice (Charles River, Germany). Mice were SC injected with saline, ghrelin, Dpr³ghrelin and selected ghrelin analogs no. 4 and 14 at a dose of 5 mg/kg of body weight (0.2 ml/mouse). 10 min after the injection, blood was collected, plasma was prepared and stored at −20° C. until use. GH levels in the plasma samples were determined by Rat/Mouse Growth Hormone ELISA kit (Merck-Millipore, St. Charles, Mo., USA). Ghrelin analogs no. 4 and 14 did not alter the GH levels, contrary to ghrelin and Dpr³ghrelin (FIG. 5).

### Example 6

### Effects of Ghrelin Analog No. 2 in Rat Model of Renal Cachexia (Subtotal Nephrectomy—SNx)

Male F344 rats (Harlan, Italy) were housed at a temperature of 22±2° C. under a daily cycle of 12/12 h light/dark (light from 6:00) with free access to water and a standard chow diet St-1 (Mlýn Kocanda, Praha, Czech Republic).

To induce renal dysfunction, 5/6 nephrectomy was performed to anesthetized 8-week-old rats (SNx group, n=14); the control group was sham-operated (Sham group, n=6). Starting from the following day, the rats were SC injected with saline or ghrelin analog no. 2 (5 mg/kg of body weight, dissolved in saline) for 15 days. Food intake and body weight of the rats was monitored every day. At the end of the experiment, blood was collected for determination of pro-inflammatory cytokines using the Milliplex MAP Rat Cytokine/Chemokine Magnetic Bead Panel (Merck-Millipore, St. Charles, Mo., USA).

Subtotal nephrectomy leads to uremia, decrease on food intake and body weight, and subsequently to cachexia. Repeated administration of analog no. 2 significantly increased body weight of SNx rats in comparison with SNx rats injected with saline (FIG. 6). Tab. 5 summarizes levels of some pro-inflammatory cytokines in plasma of the rats at the end of the experiment. Repeated administration of analog no. 2 significantly decreased levels of these cytokines in SNx rats in comparison with SNx rats injected with saline. Conversely, levels of anti-inflammatory cytokine IL-10 were significantly increased. Thus, repeated administration of analog 2 improved the cachectic state in this animal model of cachexia.

TABLE 5
Rat model of subtotal nephrectomy: levels of pro-inflammatory cytokines and IL-10 in
blood plasma after 15-day SC administration of ghrelin analog no. 2

| Compound/ | IL-1α | IL-4 | IL-2 | IL-6 | TNF-α | IL-10 |
|---|---|---|---|---|---|---|
| group | pg/ml | pg/ml | pg/mL | pg/ml | pg/ml | pg/ml |
| Saline/SNx | 584.9 ± 12.6 | 87.7 ± 12.4 | 85.4 ± 18.0 | 533.4 ± 72.7 | 55.4 ± 11.9 | 126.3 ± 5.8 |
| Analog 2/SNx | 392.3 ± 49.5* | 54.0 ± 6.4* | 28.1 ± 9.3** | 337.3 ± 62.8* | 32.6 ± 3.8* | 150.7 ± 6.2* |
| Saline/sham | 347.3 ± 51.5* | 57.8 ± 5.7 | 17.3 ± 0.6** | 285.0 ± 34.7* | 31.7 ± 4.4* | 130.6 ± 9.7 |

Values are expressed as average ± S.E.M. (n = 5-7).
*P < 0.05, **P < 0.01 vs Saline/SNx group (statistics: one-way ANOVA followed by Dunnett's post-hoc test).
SNx - rats subjected to subtotal nephrectomy,
sham - control group (sham-operated),
IL—interleukin,
TNF—tumor necrosis factor.

### Example 7

### Effects of Ghrelin Analog No. 2 in Rat Model of Aortic Stenosis-Induced Cachexia

Cachexia induced through the aortic stenosis (AS) was made in male Wistar rats, aged 8-9 months (Harlan, Italy). Rats were housed at a temperature of 22±2° C. under a daily cycle of 12/12 h light/dark (light from 6:00) with free access to water and a standard chow diet (Altromin, Germany).

The animals were anesthetized with isoflurane and the aortic stenosis was induced via abdominal incision. The abdomen was opened and abdominal aorta was surgically dissected from the inferior vena cava between the renal arteries. A steel wire (diameter 0.4 mm) was placed alongside the isolated aorta, both were gently tightened with a stitch (3-0 silk tread), and the wire was removed accordingly. The results of pilot experiments indicated that size of this diameter produced severe aortic constriction which is able to produce heart failure followed by cachexia. Finally, the abdomen was sutured and the animals were kept in standard conditions with free access to regular rat chow and water ad libitum. The Sham group consisted of sham-operated rats prepared by a similar surgical treatment without aortic stenosis.

Mortality was about 5% within 72 h after stenosis. All animal experiments were approved by the Institutional Animal Experimental Ethics Committee of the Institute of Physiology AS CR. Starting four weeks after surgery, the rats were infused intraperitoneally (using Alzet osmotic minipumps) with vehicle (PBS with 5% Tween 80) or ghrelin analog no. 2 dissolved in vehicle (2.5 mg/kg of body weight per day) for 28 days (n=5). Food intake and body weight of the rats was monitored every day.

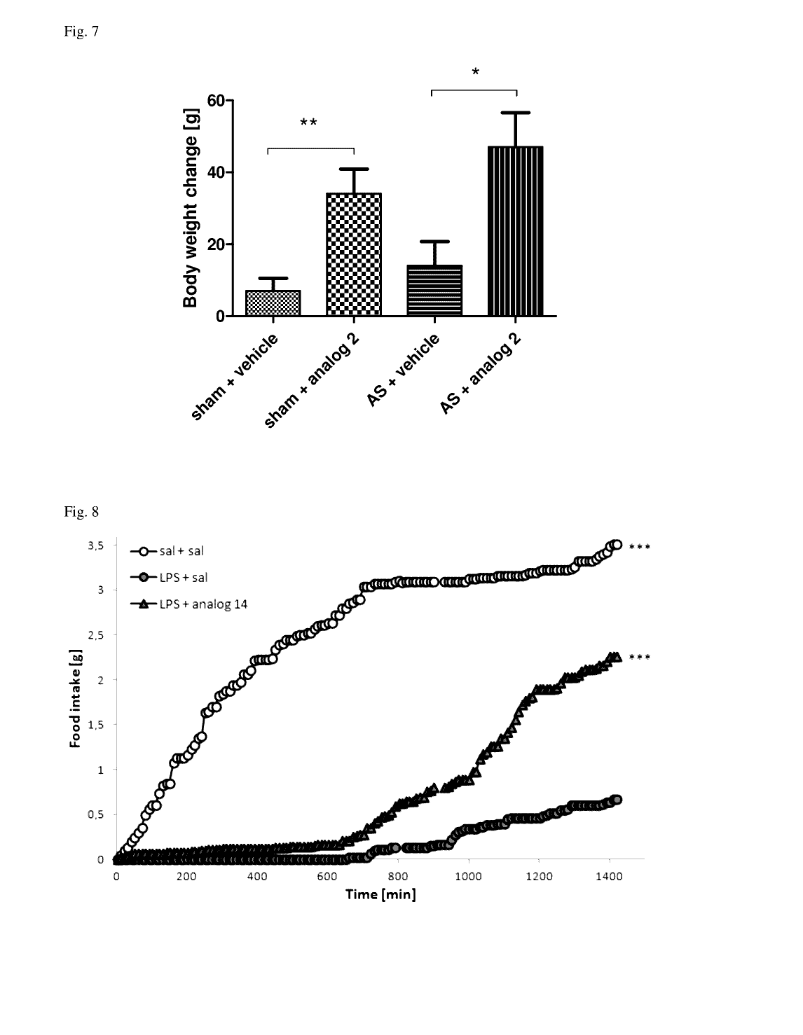

Aortic stenosis leads to decreased food intake and body weight, and subsequently after several weeks to cachexia. Intraperitoneal infusion of analog no. 2 significantly increased body weight of AS rats with stenosis in comparison with AS rats injected with saline (FIG. 7).

### Example 8

### Effects of Ghrelin Analog No. 14 in Mouse Model of Lipopolysaccharide-Induced Cachexia (LPS Model)

Male C57BL/6 mice (Charles River, Germany) were housed at a temperature of 22±2° C. under a daily cycle of 12/12 h light/dark (light from 6:00) with free access to water and a standard chow diet St-1 (Mlýn Kocanda, Praha, Czech Republic). One week before the experiment, the mice were placed into separated cages and habituated to an automatic system for food intake monitoring (Developmental workshop IOCB, Prague, Czech Republic). Animals were randomized into two groups, LPS group and control group. At 17:00, the LPS group was injected intraperitoneally (IP) with 100 μg/kg of body weight LPS (Escherichia coli 055:B5 serotype, Sigma) in a volume of 0.2 ml saline. The control group received an IP injection with an equal volume of saline. Food intake was monitored automatically in 10-min intervals. At 7:00 of the following day, the mice of both groups were SC injected with 0.2 ml of either saline or ghrelin analog no. 14 (n=4-5 mice per group). Food intake was monitored for another 10 hours.

At the end of the experiment, mice were sacrificed by decapitation, blood was collected, plasma was prepared and stored at −20° C. Hypothalamus was dissected, frozen on dry ice and stored at −80° C., musculus gastrocnemius was dissected, flash-frozen in liquid nitrogen and stored at -80° C. C-reactive protein levels and pro-inflammatory/anti-inflammatory cytokine levels in blood plasma were determined using C-Reactive Protein Mouse ELISA (Biovendor, Bmo, Czech Republic) and Milliplex MAP Rat Cytokine/Chemokine Magnetic Bead Panel (Merck-Millipore, St. Charles, Mo., USA), respectively. Samples of hypothalamus and muscle were homogenized, total RNA was extracted and RNA concentration was determined as previously described (Holubova et al., 2014). Determination of the mRNA expression of genes of interest (MuRFI and MAFbx in muscle, NPY and AgRP in hypothalamus) was performed using an ABI PRISM 7500 instrument (Applied Biosystems, Foster City, Calif., USA). The expression of GAPDH or GUSH was used to compensate for variations in input RNA amounts and the efficiency of reverse transcription, and the modified formula 2-ΔCt was used to calculate the relative gene expression.

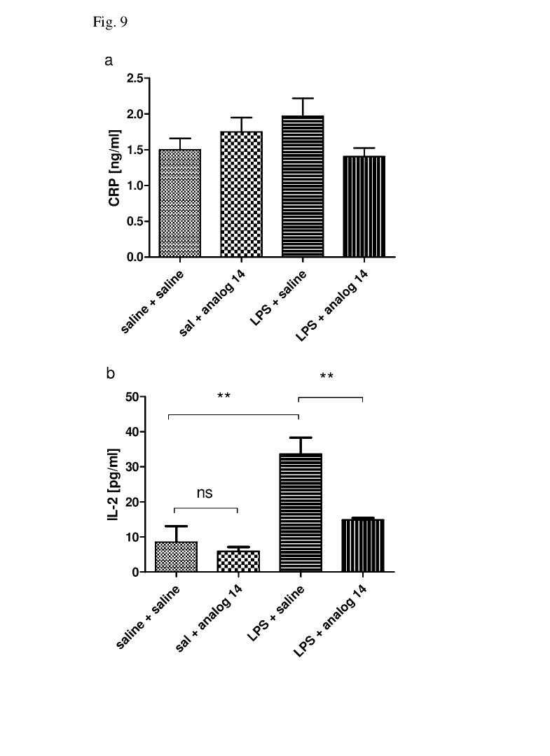

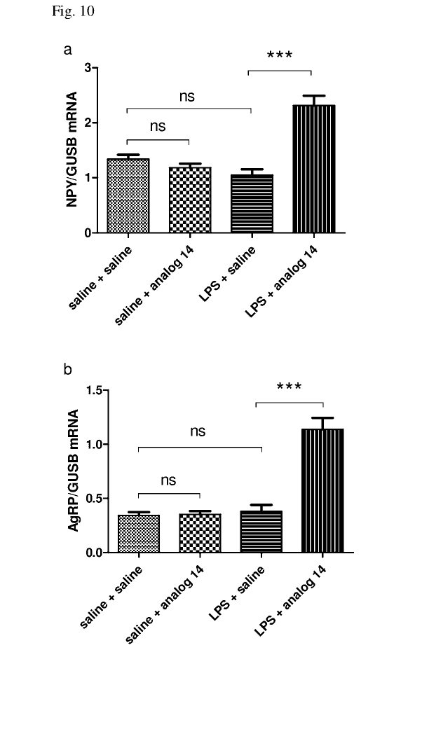

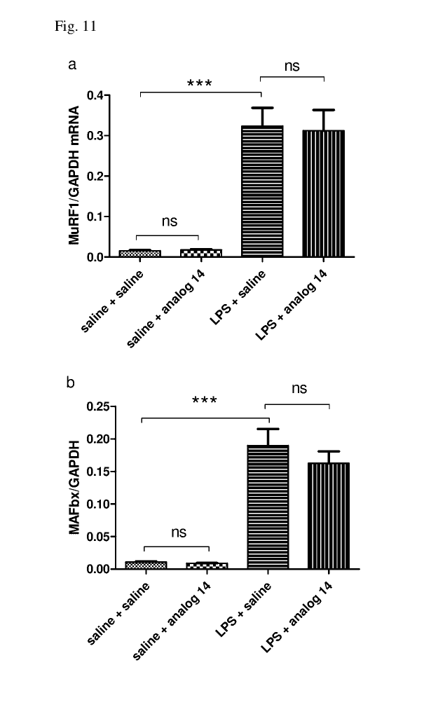

As it is apparent from FIG. 8, reduced food intake after the LPS administration and significant increase in food intake after the SC administration of analog no. 14 to the LPS model occurred. Analog no. 14 tended to normalize levels of C-reactive protein increased by the LPS and it also normalized levels of pro-inflammatory cytokine IL-2 (FIG. 9). Analog no. 14 significantly increased expression of mRNA for orexigenic neuropeptides NPY and AgRP in hypothalamus after the SC administration (FIG. 10) and thus activated orexigenic pathways. Analog no. 14 tended to decrease mRNA expression for markers of muscle degradation MuRF-1 and MAFbx which was increased after the LPS administration (FIG. 11). Thus, a single administration of analog 14 improved the cachectic state in this animal model of cachexia, increased food intake, attenuated inflammation and reduced muscle degradation.

### INDUSTRIAL APPLICABILITY

The invention is useful in pharmaceutical industry and medicine for the treatment of cachexia and/or anorexia using medicaments suitable for subcutaneous administration.

### REFERENCES

DeBoer, M. D. 2011. Mol Cell Endocrinol, 340, 97-105.

Deboer, M. D., Zhu, X., Levasseur, P. R. et al. 2008. Endocrinology, 149, 827-835.

Demange, L., Boeglin, D., Moulin, A. et al. 2007. 1. J Med Chem, 50, 1939-1957.

Depoortere, I. 2009. Regul Pept, 156, 13-23.

Garcia, J. M., Friend, J. & Allen, S. 2013. Support Care Cancer, 21, 129-137.

Holubová, M., Nagelová, V., Lacinová, Z. et al. 2014. Mol Cell Endocrinol, 393, 120-128.

Hosomi, S., Oshitani, N., Kamata, N. et al. 2008. Inflamm Bowel Dis, 14, 1205-1213.

Howard, A. D., Feighner, S. D., Cully, D. F. et al. 1996. Science, 273, 974-977.

Kojima, M., Hosoda, H., Date, Y., Nakazato, M., Matsuo, H. & Kangawa, K. 1999. Nature, 402, 656-660.

Mabulerová, J., Hlavácek, J., Blokesová, D. et al. 2007. Peptides, 28, 1945-1953.

Maletinská, L., Pýchová, M., Holubová, M. et al. 2012. J Pharmacol Exp Ther, 340, 781-786.

Matsumoto, M., Hosoda, H., Kitajima, Y. et al. 2001. Biochem Biophys Res Commun, 287, 142-146.

Motulsky, H. & Neubig, R. 2002. Curr Protoc Neurosci, Chapter 7, Unit 7.5.

Nakazato, M., Murakami, N., Date, Y. et al. 2001. Nature, 409, 194-198.

Nass, R., Pezzoli, S. S., Oliyeri, M. C. et al. 2008. Ann Intern Med, 149,601-611.

Strasser, F., Lutz, T. A., Maeder, M. T. et al. 2008. Br J Cancer, 98, 300-308.

Van Craenenbroeck, M., Gregoire, F., De Neef, P., Robberecht, P. & Petrel, J. 2004. Peptides, 25, 959-965.

Xu, X. B., Pang, J. J., Cao, J. M. et al. 2005. Am J Physiol Heart Circ Physiol, 289, H1643-1651.

1. Long-acting stable peptide ghrelin analogs of general formulae
(I)
(Sar)S(Dpr-X¹)mLSPEHQKAQQRKESKKPPA(K-Z)LQPR,
and/or
(II)
(Sar)S(Dpr-X²)FLSPEHQKAQQR(K-Z)ES,
wherein
Dpr is diaminopropionic acid,
Sar is sarcosin,
X¹ represents a fatty acid residue selected from the group comprising octanoyl, decanoyl, myristoyl, 9-decenoyl and N-10-undecynoyl bound to Dpr through an amide bond,
X² represents decanoyl or myristoyl,
m represents a non-coded amino acid selected from the group comprising phenylalanine, naphtylalanine, cyclohexylalanine, t-butylalanine and dichlorophenylalanine,
Z is palmitoyl which can be optionally bound to the secondary amino group of lysine through an amide bond or Z is not present.

2. Long-acting stable peptide ghrelin analogs of the general formulae I and/or II according to claim 1, selected from the group consisting of:
(SEQ ID NO. 4)
(Sar)S(Dpr-N-dec)FLSPEHQKAQQRKESKKPPAKLQPR
(SEQ ID NO. 5)
(Sar)S(Dpr-N-myr)FLSPEHQKAQQRKESKKPPAKLQPR
(SEQ ID NO. 6)
(Sar)S(Dpr-N-dec)(1-Nal)LSPEHQKAQQRKESKKPPAKLQPR
(SEQ ID NO. 7)
(Sar)S(Dpr-N-myr)(1-Nal)LSPEHQKAQQRKESKKPPAKLQPR
(SEQ ID NO. 8)
(Sar)S(Dpr-N-dec)(Cha)LSPEHQKAQQRKESKKPPAKLQPR
(SEQ ID NO. 9)
(Sar)S(Dpr-N-myr)(Cha)LSPEHQKAQQRKESKKPPAKLQPR
(SEQ ID NO. 10)
(Sar)S(Dpr-N-oct)FLSPEHQKAQQRKESKKPPAK(N-palm)LQPR
(SEQ ID NO. 11)
(Sar)S(Dpr-N-oct)(Cha)LSPEHQKAQQRKESKKPPAK
(N-palm)LQPR
(SEQ ID NO. 12)
(Sar)S(Dpr-N-myr)(PheCl₂)LSPEHQKAQQRKESKKPPAKLQPR
(SEQ ID NO. 13)
(Sar)S(Dpr-N-dec)FLSPEHQKAQQRK(N-palm)ES
(SEQ ID NO. 14)
(Sar)S(Dpr-N-myr)FLSPEHQKAQQRK(N-palm)ES
(SEQ ID NO. 15)
(Sar)S(Dpr-N-dec)FLSPEHQKAQQRKES

3. The long-acting stable peptide ghrelin analog according to claim 1 for use as a medicament.

4. The long-acting stable peptide ghrelin analog according to claim 1 for use in a method of treatment of cachexia and/or anorexia.

5. The long-acting stable peptide ghrelin analog according to claim 1 for use as an orexigenic compound for increasing food intake, preferably when administered by peripheral administration.

6. Pharmaceutical composition characterised in that it contains at least one long-acting stable peptide ghrelin analog according to claim 1 as an active compound, and optionally further active compounds and/or pharmaceutically acceptable auxiliary substances.
